(19) United States
(12) Patent Application Publication
(10) Pub. No.: US 2005/0214907 A1
(43) Pub. Date: Sep. 29, 2005

(54) Immunoglobulins and method for their modification

(75) Inventors: Edward Dolk, Utrecht (NL); Leo Gerardus Joseph Frenken, Vlaardingen (NL); Cornelis Theodorus Verrips, Utrecht (NL)

Correspondence Address: MORGAN LEWIS & BOCKIUS LLP, 1111 PENNSYLVANIA AVENUE NW, WASHINGTON, DC, 20004, US

(73) Assignee: Unilever Bestfoods North America, Division of Conopco, Inc.

(21) Appl. No.: 11/049,705

(22) Filed: Feb. 4, 2005

(30) Foreign Application Priority Data
Feb. 6, 2004 (EP) 04075424.4

(51) Int. Cl. C07K 16/46; C12N 5/06

(52) U.S. Cl. 435/69.1; 530/387.1; 435/326; 435/320.1

(57) ABSTRACT

Heavy chain immunoglobulins that comprise an amino acid with a side chain which is not positively charged, in the first tryptic site, especially at position 27, show increased stability in the gastrointestinal tract.

### FIELD OF THE INVENTION

The invention relates to heavy chain immunoglobulins or fragments thereof, of the VHH type with beneficial properties for use in the gastrointestinal tract.

### BACKGROUND OF THE INVENTION

Antibodies have been investigated as targeting agents for a variety of disease therapies (R. M. Reilly et al; Drug Delivery Systems, 1997; 32(4):313-323).

Several classes of antibodies exist. Among these are antibodies comprising a light chain and a heavy chain and antibodies that are devoid of the light chain.

Rotavirus is the leading etiological agent of severe diarrhea disease in infants and young children world-wide. It causes high mortality rate in developing countries and is a large economic burden in developed countries (Parashar et al, 2003) The administration of rotavirus-neutralizing bivalent (classical) antibodies can be used to modulate the infection rate of the virus. Several groups have reported the inhibition of rotavirus with antibodies (Sarker et al, 2001; Ludert et al, 2002). However, currently, there is no inexpensive treatment to reduce the severity of the infection.

A cheap alternative of using whole bivalent immunoglobulins is the use of monovalent heavy chain antibody fragments derived from llama. Expression of only the heavy chain variable part of these antibodies in plants, and lower eukaryotes, like yeast is highly efficient and results in the secretion of functional antibodies in the growth medium (Frenken et al., 2000). For such antibodies that are applied in treatment via oral administration, the stability in the gastrointestinal tract is important. Especially proteolytic stability is essential to the efficacy of the administered antibody.

Proteolytic stability is defined as the stability of proteins against the degradation by proteases such as those that exist in the stomach and in the intestines. Examples of proteases are pepsin, trypsin, chymotrypsin, carboxypeptidase and elastase. Proteolytic stability in the context of the invention relates especially to tryptic stability but also pepsin stability is envisaged.

Proteolytic stability of antibodies is for example addressed in EP-A-1318195 wherein a selected binding protein is improved to resist proteolytic degradation in a proteolytic environment. A pre-selected scaffold is subjected to random mutagenisation following which those scaffolds are selected that show improved stability towards degradation.

The resulting binding proteins are reported to be more stable but there is no information about the further properties that are desired.

Antibodies or fragments that are used in treatments further desirably show the following characteristics.

The selected and optionally mutated antibodies are preferably efficiently produced. It has been shown that fully functional VHH's can be produced efficiently in plants and in microorganisms such as a heterologous host like Saccharomyces cerevisiae. Most advantageously the antibodies are secreted into the growth medium of a microorganism as this simplifies their purification. This will enable low cost production of these compounds.

Preferably the antibodies show good binding affinity and the desired inhibition functionality under the conditions present in the G/I tract.

Preferably the antibodies are thermostable which enables their inclusion in a variety of food products. The food products may be prepared in a process comprising a heat treatment such as pasteurisation and it is preferred that the activity of the antibodies is largely maintained despite such heat treatment.

It is an object of the invention to provide antibodies or functional fragments thereof that show proteolytic stability, thermostability, good binding properties and that are easily produced in a heterologous host such as plants and microorganisms, especially lower eukaryotes.

It is a special objective to provide VHH type antibodies that can be used in treatment of rotavirus infection in an effective way.

### SUMMARY OF THE INVENTION

It has surprisingly been found that heavy chain immunoglobulins that comprise an amino acid with a side chain which is not positively charged, in the first tryptic site, especially at position 27, show increased stability in the gastrointestinal tract.

Therefore the invention relates to a method for modification of a heavy chain of an immunoglobulin which heavy chain is of VHH type, by mutation of the first tryptic site of the heavy chain molecule, wherein the mutation includes the introduction of an amino acid with a side chain which is not positively charged, in the first tryptic site.

In a further aspect the invention relates to a heavy chain immunoglobulin or fragment thereof resulting from this method, to a food product comprising such immunoglobulin and to a method for preparing such immunoglobulin or fragment.

### DETAILED DESCRIPTION OF THE INVENTION

Heavy chain of an immunoglobulin means an immunoglobulin heavy chain or a functional fragment thereof. The heavy chain immunoglobulin may be functional as such or in combination with a light chain.

The heavy chain of an immunogobulin may be naturally occurring or may be obtained by genetic engineering techniques. It is preferred that the heavy chain is selected via genetic technologies in contrast to a naturally occurring heavy chain immunoglobulin molecule.

Functional fragment of a heavy chain means a fragment of a heavy chain of an immunoglobulin, which fragment shows binding affinity for an antigen. Binding affinity is present when the dissociation constant is more than 10 exp-7.

Preferred fragments are variable domain fragments of a heavy chain of an immunogobulin, which fragments lack the constant domains.

A heavy chain of an immunoglobulin of the VHH type refers to heavy chain immunoglobulins such as those derived from the family of Camilidae. These immunoglobulins are characterised by a functional single domain antibody fragment derived from the variable heavy chain domain of camelid antibodies.

Furthermore they are often referred to as a unique sub class of antibodies lacking light chains (Hamers-Casterman C. et al. 1993, Naturally occurring antibodies devoid of light chains, Nature 363: 446-448), and consequently have only a single binding domain which is referred to as VHH.

Genetically modified means that the amino acid sequence referred to is non-naturally occurring but obtained by protein engineering techniques. The genetically modified sequences may be recognized by techniques that involve analysis of codon usage.

Amino acid position numbering in this specification and claims is based on the numbering introduced by Kabat E. A. (1991); Proteins of immunological interest, National Institute of Health Publication No. 91-3242, Bethesda, Maryland.

The term mutation refers to random or targeted genetic modification of a protein which is e.g. the deletion or change of an amino acid in a protein molecule. The terms mutation and modification are used interchangeably.

Thermostability is determined by the method exemplified in the examples, and involves the determination of Tm which is the temperature at which 50% of the protein is unfolded.

Proteolytic stability refers to stability of a protein against degradation by proteolytic enzymes. The method to determine proteolytic stability against trypsin is described in the examples and comprises treatment with trypsin and measurement of remaining functionality and protein degradation. The tryptic degradation products are determined by techniques such as gel-electrophoresis, mass spectrophotometry or HPLC.

The method according to the invention involves mutation of the first tryptic site of a heavy chain molecule. In this context the first tryptic site is the site which is first attacked by trypsin. It was found that there was a correlation between accessible surface area and probability of cleavage. The first cleavage site had the highest accessible surface area. The first tryptic site is the site which is kinetically first attacked by trypsin and hence is most vulnerable to cleavage. It was surprisingly found that the modification of this site leads to increased tryptic stability in spite of many other positively charged amino acids that are present on the surface of the VHH, whereas the resulting compounds could still be produced as efficiently as the wild type VHH in a host system such as yeast, were even more thermostable and retained their binding characteristics to a desirable level.

In a further aspect the invention relates to a method to improve the proteolytic stability of a heavy chain of an immunoglobulin of the VHH type by introducing on amino acid position 27 of the heavy chain, an amino acid with a side chain which is not positively charged.

In a further aspect the invention relates to a heavy chain immunoglobulin or fragment thereof of the VHH type, comprising in position 27 an amino acid with a side chain which is not positively charged.

The preferred embodiments listed for the method of producing such heavy chain immunogobulin are also applicable to this immunoglobulin or fragment thereof.

The below mentioned preferred embodiments are applicable for both the modification of the first tryptic site and the modification of the amino acid at position 27 of the heavy chain immunoglobulin.

The modification includes the introduction of an amino acid with a side chain which is not positively charged in the first tryptic site. This modification was found to achieve the above-indicated objectives.

More preferred the mutation is such that the amino acid with a side chain that is not positively charged replaces an amino acid with a basic side chain such as lysine and arginine. Most preferred the amino acid with a side chain that is not positively charged replaces an arginine in the first tryptic site, more preferably at position 27.

The amino acid with the side chain that is not positively charged, is preferably an uncharged amino acid, more preferably an amino acid with a non polar side chain. Most preferred the amino acid is selected from the group comprising alanine, glycine, serine, histidin, valine, leucine, isoleucine, phenylalanine and proline, more preferred from the group comprising alanine, glycine, histidin and serine. Most preferred the amino acid with a side chain that is not positively charged is alanine.

In a more preferred embodiment the heavy chain immunogobulin is a fragment which is devoid of a CH1 domain.

Most preferred the heavy chain immunogobulin or fragment thereof shows binding affinity with a dissociation constant of at least 10 exp.-7, preferably between 01 exp-7 and 10 exp-8 for rotavirus, especially rotavirus strains Wa, CK5, Wa1, RRV or CK5.

Even more preferred the heavy chain immunogobulin has the amino acid sequence according to SEQ ID 1, or is a functional homologue thereof that comprise alanine at position 27 and show at least 80% amino acid identity, more preferred at least 90%, most preferred at least 95% with SEQ ID 1.

Functional homologues are immunogobulin heavy chain molecules or fragments thereof that show at least 80% of the neutralisation activity of the protein according to SEQ ID 1.

In a further aspect the invention relates to a pharmaceutical preparation or food composition comprising a heavy chain immunoglobulin which contains in position 27 an amino acid with a side chain which is not positively charged.

In this embodiment the pharmaceutical preparation or food composition comprises a genetically modified heavy chain immunoglobulin according to the invention or a naturally occurring heavy chain immunoglobulin comprising in position 27 an amino acid with a side chain that is not positively charged. These antibodies were found to be especially suitable for use in pharmaceutical or food products because they can be produced in high amounts against a low price, they are thermostable, and are not readily digested by pepsin and trypsin which helps them survive G/I tract conditions. These properties make these antibodies highly suitable for use in food compositions which are usually degraded in the stomach and other parts of the G/I tract. It is important for the claimed antibodies to survive such conditions such that they are effective in any stage in the G/I tract. This is especially the case where the aim of using the antibody is neutralisation of a virus such as rotavirus which is present in the G/I tract.

The invention especially relates to a pharmaceutical preparation or food composition comprising a heavy chain immunoglobulin according to the invention or a naturally occurring heavy chain immunoglobulin comprising in position 27 an amino acid with a side chain which is not positively charged, which immunoglobulin at least partly neutralizes rotavirus infection.

Suitable food products are e.g. drinks including powdered drinks that can be reconstituted, spreadable products such as margarine and butter, rice, bread. The preferred food products are drinks such as milk and rice.

The invention also relates to polynucleotide sequences encoding the heavy chain immunoglobulin or functional fragment thereof according to the invention and to a recombinant DNA vector able to direct the expression of said nucleotide sequence.

Suitable expression systems and hosts are disclosed in EP-B-698097.

In another aspect the invention relates to a method for preparing a heavy chain immunoglobulin or fragment thereof, of the VHH type with increased tryptic stability, comprising the steps of:

a) selecting said heavy chain immunoglobulin with desired binding affinity under conditions comprising low pH and in the presence of pepsin

b) modifying at least one of said heavy chain immunoglobulins using protein engineering techniques to increase tryptic stability.

This combination of selection of an antibody molecule under specific conditions of low pH which is preferably from 1.5 to 3.5 and in the presence of pepsine, which is a protease that is abundantly present in the stomach, and subsequent modification by protein engineering, results in the claimed highly beneficial molecules that perform well in the G/I tract.

More preferably the protein engineering techniques comprise the method of directed mutation as described above.

According to a further preferred embodiment, the conditions in step (a) or the genetic engineering in step (b) are such that the resulting antibody molecules also show increased stability against cleavage by chymotrypsin.

The protein engineering techniques are preferably based on molecular modeling to investigate the most accessible tryptic site.

The selection according to step (a) is preferably carried out using yeast display, phage display and other display techniques and biopanning techniques that are well known in the art.

The invention is illustrated by the following non-limiting examples.

### EXAMPLES

1.1 Strains and Growth Media

The Escherichia coli strain used in this study was E. coli TG1 (F′ traD36 lacI^{q} Δ[lacZ]M15 proA⁺B⁺/supE Δ(hsdM-mcrB)5 [rₖ⁻mₖ⁻McrB⁻] thi Δ[lac-proAB]). This strain was grown in 2TY supplemented with 100 μg ampicillin/ml and/or 25 μg Kanamycin/ml and/or 1% glucose (final concentration, v/v) when appropriate. The Saccharomyces cerevisiae strain VWK18gal1 (CEN-PK102-3A, MATa, leu2-3, ura3, gal1:URA3, MAL-8, MAL3, SUC3) was used as a host for yeast expression studies. S. cerevisiae was grown on 2% (w/v) yeast extract, 1% (w/v) peptone and 2% (w/v) glucose (YPD) or on 0.67% (w/v) Yeast Nitrogen Base without amino acids and 2% (w/v) glucose (YNB) with appropriate amino acids added. Solid media contained, in addition to YNB, 2% (w/v) agar.

Bovine Rotavirus Compton CK5 was obtained from the Moredun Institute, Midlothian, Scotland and the BS-C1 Cells were purchased from the European Animal Cell Culture Collection. CK5 Rotavirus seed was activated by incubating seed diluted 1:10 in Serum Free Medium (SFM) EMEM supplemented with 1% MEM Amino Acids solution (100×), 20 mmol 1⁻¹ L-Glutamine and 0.5 μg/ml crystalline trypsin for one hour at 37° C. After activation the seed was further diluted in SFM containing 0.5 μg/ml crystalline trypsin and then 5 ml of diluted seed was added to confluent monolayers of BS-C1 cells in 162 cm² tissue culture flasks. The virus was adsorbed on to the cells for one hour at 37° C. then the medium was topped up to 75 ml. The bottles were incubated at 37° C. until complete cytopathic effect was observed. Cultures were frozen (−70° C.) and thawed twice, then pooled and centrifuged at 1450 g for 15 minutes at 4° C. to remove cell debris. The supernatant was decanted and stored in aliquots at −70° C.

The BS-C1 cells were cultured in Earles Modified Essential Medium supplemented with 10% Heat inactivated foetal calf serum, 1% MEM Amino Acids solution (100×), 20 mmol 1⁻¹ L-Glutamine, 100 I.U ml⁻¹ penicillin, 100 μg ml⁻¹ streptomycin and 2.5 μg ml⁻¹ amphotericin B. The culturing conditions were as previously decribed by Johansen et al., Vaccine 2003 Jan 17; 21 (5-6): 368-75.

1.2 Reagents YNB, Bacto Peptone, Bacto Yeast Extract and Bacto Agar were from

Difco Laboratories (Detroit, USA). DNA restriction and modification enzymes were from New England Biolabs, Inc. (Beverly, USA) and Boehringer Mannheim Biochemicals (Indianapolis, Ind.). Microcon 30 microconcentrators were from Amicon (Beverly, USA; Millex-HA 0.45 μm filter units were from Millipore Corp. (Bedford, USA). The goat anti-rabbit antibody was purchased from Bio Rad Laboratories (Hercules, USA) and the anti-mouse HRP conjugate from DAKO (Glostrup Denmark). Anti-6xHis-HRP antibody conjugate was from Roche Molecular (Pleasanton, USA). MEM Amino Acids solution (100×), L-Glutamine, crystalline trypsin, Earles Modified Essential Medium, Penicillin, Streptomycin and Amphotericin B were purchased from Sigma (St. Louis, USA). Tissue culture flasks were purchased from Costar (Bucks, UK).

1.3. Selection of Rotavirus Specific Heavy-Chain Antibodies from a Llama Immune Phase Display Library.

1.3.1. Immunisation

A llama was immunized subcutaneously and intramuscularly at day 0, 42, 63, 97 and 153 with 5×10¹² pfu (approx. 100 μg protein) of rhesus-monkey rotavirus serotype G3, strain RRV. This rotavirus strain was purified, amplified and concentrated as described previously by Johansen et al., Vaccine 2003 Jan 17; 21 (5-6): 368-75.

Prior to immunization, the viral particles were taken up in oil emulsion (1:9 V/V, antigen in PBS : Specol (Bokhout et al. 1981, 1986) as described before (Frenken et al., 2000). The immune response was followed by titration of serum samples in ELISA with RRV rotavirus coated at a titer of 4×10⁶ pfu/ml in 0.9% NaCl following the protocol described before (de Haard et al., 1999; Frenken et al., 2000).

1.3.2. cDNA Amplication

An enriched lymphocyte population was obtained from the 153-day blood sample of about 150 ml via centrifugation on a Ficoll (Pharmacia) discontinuous gradient. From these cells, total RNA (between 250 and 400 μg) was isolated by acid guanidium thiocyanate extraction (Chomczynnski and Sacchi, 1987). Subsequently, first strand cDNA was synthesized using the Amersham first strand cDNA kit (RPN1266). In a 20 μl reaction mix 0.4-1 μg mRNA was used. The 6-mer random primer was used to prime the first DNA strand. After cDNA synthesis, the reaction mix was directly used for amplification by PCR. V_{H}H genes were amplified with primers
Lam-16 (GAGGTBCARCTGCAGGASAGYGG),
Lam-17 (GAGGTBCARCTGCAGGASTCYGG),
Lam-07 (priming to the short hinge region) and
Lam-08 (long hinge specific) (Frenken et al., 2000).

Amplification of DNA was performed as described by De Haard et al (1999).

1.3.3. Phage Display

The amplified products were digested with PstI and NotI and cloned in phagemid vector pUR5068, which is identical to pHEN1 (Hoogenboom et al., 1991), but containing a hexahistidine tail for Immobilized Affinity Chromatography (Hochuli et al., 1988) and a c-myc derived tag (Munro and Pelham, 1986) for detection. Ligation and transformation were performed as was described before (de Haard et al., 1999).

The rescue with helperphage VCS-M13 and PEG precipitation was performed as described before (Marks et al., 1991).

1.3.4. Selection Process

Selections were performed via the biopanning method (Marks et al., 1991) by coating of Rotavirus strain RRV (2.5×10⁷ pfu/ml at round 1; 5×10⁴ pfu/ml at round 2; 500 pfu/ml at round 3 and 50 pfu/ml at round 4). Viral particles were captured via polyclonal anti-rotavirus sera. Therefore, Immunotubes (Nunc, Roskilde, Denmark) were coated overnight at 4° C. with either a 1:1000 dilution of anti-rotavirus rabbit sera or anti-rotavirus guinea pig sera in carbonate buffer (16% (v/v) 0.2 M NaHCO₃+9% (v/v) 0.2 M Na₂CO₃). In addition to the standard selections, the antibody fragment displaying phages have been pre-incubated with acid and pepsin. This was done by incubating phages in a dilute HCl solution (pH 2.3) with or without 25 U of pepsin at 37° C. for 15 min. The pepsin activity was inhibited by the addition of 10 μl of pepstatin A (1 mg/ml in ethanol). After this incubation, the standard selection procedure was followed.

1.3.4. Screening for Heavy-Chain Antibodies with Specifically High Affinity for Rotavirus

Soluble VHH was produced by individual E. coli clones as was described (Marks et al., 1991). Culture supernatants were tested in ELISA. Microlon F (Greiner Bio-One GmbH, Germany) plates were coated with 50 μl / well of a 1:1000 dilution of either anti-rotavirus rabbit polyclonal sera or anti-rotavirus guinea pig polyclonal sera in carbonate buffer (16% (v/v) 0.2 M NaHCO₃+9% (v/v) 0.2 M Na₂CO₃) and subsequently incubated with rotavirus strain CK5 (approx. 109 pfu/ml). After incubation of the VHH containing supernatants, VHH's were detected with a mixture of the mouse anti-myc monoclonal antibody 9E10 (500 ng/ml) and anti-mouse HRP conjugate (250 ng/ml). Alternatively, detection was performed with anti-6xHis-HRP antibody conjugate (1000 ng/ml). Fingerprint analysis (Marks et al., 1991) with the restriction enzyme HinFI was performed on all clones.

1.4. Antibody Fragment Production in Saccharomyces cerevisiae.

A set of rotavirus-specific antibody fragments were selected. DNA sequences encoding these antibody fragments were isolated from pUR5068 (PstI/BstEII) and cloned into pUR4547 which is identical to the previously described pUR4548 (Frenken et al., 2000), but does not encode any C-terminal tag-sequences. This episomal yeast expression vector contains the GAL7 promoter, the SUC2 signal sequence for high level expression and secretion into the growth medium, respectively. The Saccharomyces cerevisiae strain VWK18gal1 was a gall derivative of CEN.PK102-3A (MATa leu2 ura3) obtained by disruption of the GALL gene by integration of the S. cerevisiae URA3 gene (Rothstein, 1983) This strain was transformed and induced for antibody fragment production as described previously in EP-B-698097 and by vd Vaart et al, 2002.

1.5 Homology Modelling

The structure of VHH1 was modeled based on a multiple alignment of sequences from all available PDB structures and VHH1 sequence using ClustalW. The model was build using the WHAT IF software (Vriend, 1990) based on the structure of the VHH directed against RR6, PDB entry 1QD0 (Spinelli, 2000), as described previously (Vriend, 1993). Modelling details are available at http://www.cmbi.kun.nl/articles.ext/.

1.6 Mutant Design

The accessibility of the arginine and lysine residues in VHH1 was determined using the surface-accessibility module of WHAT IF (Vriend, 1990). The compliance with the serine protease specificity rules of Frenken was determined as described before (Frenken, 1993). Multiple alignments were performed with 703 public and proprietary sequences of VHHs as obtained from previous unrelated studies on llama antibody fragments. All numbering and CDR definitions are according to Kabat (Kabat, 1994).

1.7 Construction of the Mutants

Mutants of VHH1 were created with PCR by ‘splicing by overlap extension’. In short, complementary primers, with nucleotides substituted for generating the desired mutation are used in a two-step PCR to generate DNA fragments containing the desired mutation. These products were digested with restriction enzymes and cloned into vector pUR 4585 containing a Myc-tag and a His-tag (Frenken, 2000).

1.8 Trypsin Digestion

Eight pg of VHH was incubated with 20 μl immobilized TPCK trypsin (Pierce) in PBS at 37° C. After 0, 15, 45 and 90 minutes of head-over-head incubation, 10 μl samples were taken. Immediately protein sample buffer was added and the samples were boiled for 5 min. Samples were stored at −20° C. until all samples were collected, and subsequently separated with SDS PAGE.

1.9 Determination of T_{M}

CD spectra were measured with a Jasco J-810 spectropolarimeter coupled to a Jasco CDF-426S Peltier thermostatted cell holder. Molecular ellipticity at 200 nm was determined from 25° C. to 80° C. The spectral bandwidth was automatically kept at 2 nm, the temperature increment was 0.2 C/step and the accumulation time was 2C/min. 240 μg/ml VHH was used in a 0.2 mm quarz cuvette (Hellma). The spectra manager software (Jasco) was used to analyze the spectra. Typically, the spectra of the VHHs were smoothened and the first derivative was determined.

1.10 Functional Assay

Determination of the binding capacity of the mutants was performed in a competition ELISA based assay. UV-inactivated bovine (Compton) rotavirus strain G3 CK5 was coated onto maxisorp plates. Plates were blocked for 30 minutes with 4% MPBST (4% Marvel in PBS with 0.05% Tween20). Fifty pl of 250 ng/ml VHH1-biotin in 2% MPBST was added to 50 μl of competition samples. Competition samples contained VHH1 or the mutants. The highest concentration of 50 μg/ml and samples were diluted with 2% MPBST. Subsequently this mixture of biotinylated VHH1 and competitor were incubated for one hour. After washing, streptavidine-HRP (1:1000 in 2% MPBST) was added to all wells and incubated for one hour. Subsequently, the plate was washed and HRP activity was determined with TMB substrate (Biomerieux, Boxtel). The color reaction was stopped with 50 μl H₂SO₄ and measured at 450 nm in a plate reader.

Determination of the binding capacity after trypsin digestion was performed using the same assay. Trypsin digestion was performed with 25 μg VHH dissolved in 100 μl PBS with 50 μl trypsin beads (Pierce). After 0, 90, 180 minutes and overnight samples of 15 μl were taken, to which 5 μl soy bean trypsin inhibitor (5 μg/μl) was added. These VHH samples were diluted to 50 μg/ml in 2% MPBST.

2. Results

The following mutants were constructed:
R27A (SEQ ID 1)
R27F (SEQ ID 2)

They were compared to a VHH of SEQ ID 3 which contains a positively charged amino acid (27R) in the first tryptic site on position 27.

The two mutant proteins were subjected to trypic digestion. As the second amino acid of the Myc-tag is a lysine, the tags are likely to be very flexible and thus susceptible to tryptic cleavage

A coomassie stained gel of the resulting digests for a non modified VHH with R at position 27 showed three bands. The upper band was the intact VHH with the Myc-His tag, the middle band was the intact VHH without the tags. This was confirmed by Western blot with anti-his and anti-VHH. The third band was a VHH degradation product. The mutants R27F and R27A did not show this third band, indicating that these mutants were not cleaved. The size of the degradation product was about 12 kD, which corresponds well with VHH of sequence ID 1 with 27R, without the first 27 amino acids. Position 27 of unmodified VHH according to SEQ id 1 with he modification that position 27 is R is thus the first tryptic cleavage site. And

After 45 minutes, 50% of the wild-type (27R) is degraded, and after 180 minutes only very little VHH is left. Mutant R27A according to sequence ID 1 shows complete removal of the tags after 15 minutes. No significant further degradation of R27A was seen up to 24 hours.

Binding affinity competition experiment. 250 ng/ml biotinylated VHH according to SEQ ID 1 and varying concentrations (as indicated on the horizontal axis) of non-biotinylated variants compete for rotavirus binding. OD450 is related to binding of biotinylated VHH.

A competition ELISA assay was performed to check that the mutants were still able to bind rotavirus, and to estimate their binding affinities. Variants R27A (SEQ ID 1) and R27F (SEQ ID 2) showed binding affinities comparable to wild-type (SEQ ID 3), except that R27F, showed a decrease in affinity but was still active.

The rotavirus-binding capacity of wild-type VHH with 27R and of R27A as a function of tryptic digestion was determined. This showed that after prolonged digestion, the rotavirus-binding capacity of the wild-type decreases. R27A, according to sequence ID 1, neither gets cleaved, nor looses its rotavirus-binding capacity under these conditions. So, the partially cleaved VHH of SEQ ID 1 is still capable of binding rotavirus, and it can thus be concluded that the R27A mutation is important because it prevents subsequent cleavage at other sites.

Thermal stability of proteins often is also a good indicator for stability against a series of irreversibly inactivating conditions, like trypsin, pepsin and low pH mutants were designed with avoidance of proteolysis as the major constraint while stability considerations were minor.

The VHH according to SEQ ID 1 was produced in S. Cerevisiae according to the method disclosed by vd Vaart et, 2002. The VHH could be produced in at least equal amounts and with the same efficiency as the wild type VHH (27R; SEQ ID 3).

The above confirms that the modified VHH type heavy chain immunoglobulins which are according to the invention, maintain their binding affinity, have increased stability towards tryptic digestion, are thermostable and can be produced in yeast in high amounts.

### LIST OF REFERENCES

Bokhout, B. A., Van Gaalen, C., and Van Der Heijden, Ph. J., (1981), A selected water-in-oil emulsion: composition and usefulness as an immunological adjuvant. Vet. Immunol. Immunopath., 2: 491-500
Bokhout, B. A., Bianchi, A. T. J., Van Der Heijden, Ph. J., Scholten, J. W. and Stok, W., (1986), The influence of a water-in-oil emulsion on humoral immunity. Comp. Immun. Microbiol. Infect. Dis., 9: 161-168.
Chomczynski,P. and Sacchi,N. (1987) Single-step method of RNA isolation by acid guanidinium thiocyanate- phenol-chloroform extraction. Anal. Biochem., 162, 156-159.
Cook, S. M., Glass, R. I., Lebaron, C. W. and Ho, M. S. 1990. Global seasonality of rotavirus infections. Bulletin of the world health organization 68: (2) 171-177.
de Haard,H. J., van Neer,N., Reurs,A., Hufton,S. E., Roovers,R. C., Henderikx,P., de Bruine,A. P., Arends,J. W. and Hoogenboom,H. R. (1999) A large non-immunized human Fab fragment phage library that permits rapid isolation and kinetic analysis of high affinity antibodies. J. Biol. Chem., 274, 18218-18230.
Espinoza, F., Paniagua, M., Hallander, H., Svensson, L. and Strannegard, O. 1997. Rotavirus infections in young nicaraguan children. Pediatric infectious disease journal 16:564-571.
Frenken,L. G., van der Linden,R. H., Hermans,P. W., Bos,J. W., Ruuls,R. C., de Geus,B. and Verrips,C. T. (2000) Isolation of antigen specific llama VHH antibody fragments and their high level secretion by Saccharomyces cerevisiae. J. Biotechnol., 78, 11-21.
Glass, R. I., Lew, J. F., Gangarosa, R. E., Lebaron, C. W. and Ho, M. S. 1991. Estimates of morbidity and mortality-rates for diarrheal diseases in american children. Journal of pediatrics 118: (4) s27-s33, part 2.
Guarino, A, Guandalini, S., Albano, F., Mascia, A., Deritis, G.and Rubino, A. 1991. Enteral immunoglobulins for treatment of protracted rotaviral diarrhea. Pediatric infectious disease journal 10:612-614.
Hamers-Casterman,C., Atarhouch,T., Muyldermans,S., Robinson,G., Hamers,C., Songa,E. B., Bendahman,N. and Hamers,R. (1993) Naturally occurring antibodies devoid of light chains. Nature, 363, 446-448.
Hanson, L. A., Hahn-Zoric, M. and Berndes, M. 1994. Breast feeding: Overview and breast milk immunology. Acta Paediatr. Jpn. 36:557-661.
Hochuli,E., Bannwarth,W., Dobeli,H., Gentz,R. and Stuber,D. (1988) Genetic approach to facilitate purification of recombinant proteins with a novel metal chelate adsorbent. Bio/Technol. 6, 1321-1325.
Hoogenboom,H. R., Griffiths,A. D., Johnson,K. S., Chiswell,D. J., Hudson,P. and Winter,G. (1991) Multi-subunit proteins on the surface of filamentous phage: methodologies for displaying antibody (Fab) heavy and light chains. Nucleic Acids Res., 19, 4133-4137.
Laemmli, U. K. 1970. Cleavage of structural proteins during assembly of the head bacteriophage T4. Nature 227:680-685.
Marks,J. D., Hoogenboom,H. R., Bonnert,T. P., McCafferty,J., Griffiths,A. D. and Winter,G. (1991) By-passing immunization: Human antibodies from V-gene libraries displayed on phage. J. Mol. Biol., 222, 581-597.
Munro,S. and Pelham,H.R. (1986) An Hsp70-like protein in the ER: identity with the 78 kd glucose- regulated protein and immunoglobulin heavy chain binding protein. Cell, 46, 291-300.
Offit, P. A. and Clark, H. F. 1985. Protection against rotavirus-induced gastroenteritis in a murine model by passively acquired gastrointestinal but not circulating antibodies. Journal of virology 54:58-64.
R. M. Reilly et al; Drug Delivery Systems, 1997; 32(4):313-323
Rothstein,R. J. (1983) One-step gene disruption in yeast. Methods Enzymol, 101, 202-211.
Schibler,U., Marcu,K. B. and Perry,R. P. (1978) The synthesis and processing of the messenger RNAs specifying heavy and light chain immunoglobulins in MPC-1l cells. Cell, 15, 1495-1509.
Sarker, S. A., Casswall, T. H., Mahalanabis, D., Alam, N. H., Albert, M. J., Brussow, H., Fuchs, G. J. and Hammerstrom, L. 1998. Successful treatment of rotavirus diarrhea in children with immunoglobulin from immunized bovine colostrum. Pediatric infectious disease journal 17: 1149-1154.
van der Vaart J M. Expression of VHH antibody fragments in Saccharomyces cerevisiae. Methods Mol Biol. 2002;178:359-66.

1. Method for modification of a heavy chain of an immunoglobulin which heavy chain is of VHH type, by mutation of the first tryptic site of the heavy chain molecule, wherein the mutation includes the introduction of an amino acid with a side chain which is not positively charged, in the first tryptic site.

2. Method to improve the proteolytic stability of a heavy chain of an immunoglobulin of the VHH type by introducing on amino acid position 27 of the heavy chain, an amino acid with a side chain which is not positively charged.

3. Method according to claim 1 wherein the amino acid that is introduced is an uncharged amino acid, preferably an amino acid with a non polar side chain.

4. Method according to claim 1, wherein the amino acid that is introduced is selected from the group comprising alanine, glycine, histidin, phenylalanine, serine, valine, leucine and isoleucine, and proline, more preferred alanine, glycine, histidin and serin.

5. Method according to claim 1 wherein the introduced amino acid is alanine.

6. Method according to claim 1 wherein the introduced amino acid replaces an arginine or lysine.

7. Method according to claim 1 wherein the heavy chain immunoglobulin is a fragment, devoid of a CH1 domain.

8. Genetically modified heavy chain immunoglobulin or fragment thereof of the VHH type, comprising in position 27 an amino acid with a side chain which is not positively charged.

9. Genetically modified heavy chain immunoglobulin or fragment thereof according to claim 8, containing in position 27 an amino acid selected from the group comprising alanine, glycine, phenylalanin, histidin, serine valine, leucine, isoleucine, and proline.

10. Genetically modified heavy chain immunoglobulin or fragment thereof according to claim 8 wherein the amino acid at position 27 is alanine.

11. A pharmaceutical preparation or food composition comprising a heavy chain immunoglobulin according to claim 8 or a naturally occurring heavy chain immunoglobulin of the VHH type comprising in position 27 an amino acid with a non positively charged side chain.

12. A pharmaceutical preparation or food composition comprising a heavy chain immunoglobulin which contains in position 27 an amino acid with a side chain which is not charged.

13. Polynucleotide sequences encoding the heavy chain immunoglobulin or functional fragment thereof according to claim 8.

14. A method for selecting heavy chain immunoglobulins or fragments thereof of the VHH type with increased pepsin and tryptic stability, comprising the steps of:
a) selection of said heavy chain immunoglobulin with desired binding affinity under conditions comprising low pH and the presence of pepsin
b) modification of at least one of said heavy chain immunoglobulins using protein engineering techniques to increase tryptic stability.

15. Method according claim 14 wherein the protein engineering techniques comprise mutation of the first trvptic site of the heavv chain molecule, wherein the mutation includes the introduction of an amino acid with a side chain which is not positively charged, in the first trvptic site.


SEQUENCE LISTING (5 sequences)

SEQ ID NO: 1 - protein, 123 aa, Artificial Sequence
  VHH with good binding affinity for rotavirus with modification R27A. Sequence based on VHH derived from llama
QVQLQDSGGGLVQVGDRLSLSCAASGATFSSYDMAWFRQAPGKEREFVAAITTSEGTWYGDAGKGRFTIARVNAKNTVYLHMNRLKPEDTAVYYCAASNQGGSLQISTNYNYWGQGTQVTVSS

SEQ ID NO: 2 - protein, 123 aa, Artificial Sequence
  VHH with good binding affinity for rotavirus with modification R27F. Sequence based on VHH derived from llama.
QVQLQDSGGGLVQVGDRLSLSCAASGFTFSSYDMAWFRQAPGKEREFVAAITTSEGTWYGDAGKGRFTIARVNAKNTVYLHMNRLKPEDTAVYYCAASNQGGSLQISTNYNYWGQGTQVTVSS

SEQ ID NO: 3 - protein, 123 aa, Artificial Sequence
  VHH with affinity for rotavirus, derived from llama
QVQLQDSGGGLVQVGDRLSLSCAASGRTFSSYDMAWFRQAPGKEREFVAAITTSEGTWYGDAGKGRFTIARVNAKNTVYLHMNRLKPEDTAVYYCAASNQGGSLQISTNYNYWGQGTQVTVSS

SEQ ID NO: 4 - DNA, 23 bp, Artificial Sequence
  PCR primer for VHH with affinity for rotavirus, derived from llama
gaggtbcarctgcaggasagygg

SEQ ID NO: 5 - DNA, 23 bp, Artificial Sequence
  PCR primer for VHH with affinity for rotavirus, derived from llama
gaggtbcarctgcaggastcygg